(19) United States
(12) Patent Application Publication
(10) Pub. No.: US 2024/0175804 A1
(43) Pub. Date: May 30, 2024

(54) GAS SENSOR FOR DETERMINING THE CONCENTRATION OF AT LEAST ONE GAS IN A GAS MIXTURE AND METHOD FOR DETERMINING THE CONCENTRATION OF AT LEAST ONE GAS IN A GAS MIXTURE WITH A GAS SENSOR

(71) Applicant: Endress+Hauser Optical Analysis, Inc., Ann Arbor, MI (US)

(72) Inventors: Tobias Meinert, Freiburg (DE); Benjamin Scherer, Oberried (DE); Marc Winter, Gelnhausen (DE); Valentin Wittstock, Freiburg (DE)

(21) Appl. No.: 18/522,602

(22) Filed: Nov. 29, 2023

(30) Foreign Application Priority Data
Nov. 29, 2022 (DE) 10 2022 131 508.8

(51) Int. Cl. G01N 21/17; G01N 33/00

(52) U.S. Cl. CPC G01N 21/1702; G01N 33/0027; G01N 2021/1704

(57) ABSTRACT

A gas sensor for determining a concentration of at least one gas in a gas mixture includes: at least one intensity modulatable light source; a measuring section, into which the gas mixture to be investigated can be allowed to flow; and an essentially gas-sealed detection cell, wherein the gas sensor is embodied such that light emitted from the light source is radiated into a measuring section, wherein the intensity of the emitted light is modulated with a modulation frequency, which differs from the resonant frequency of a mode of the acoustic resonance of the detection cell by less than 0.5 times, especially less than 0.25 times, the half-width of the mode. Further, a method for determining the concentration of the at least one gas in the gas mixture uses the gas sensor.

### CROSS-REFERENCE TO RELATED APPLICATION

The present application is related to and claims the priority benefit of German Patent Application No. 10 2022 131 508.8, filed Nov. 29, 2022, the entire contents of which are incorporated herein by reference.

### TECHNICAL FIELD

The present disclosure relates to a gas sensor for determining concentration of at least one gas in a gas mixture and method for determining concentration of at least one gas in a gas mixture with a gas sensor.

### BACKGROUND

Conventional photoacoustic gas sensors rely on the measuring principle that gas molecules are excited to execute mechanical (e.g., molecular) oscillations by intensity modulated radiation of an emitter unit, for example, a light source or a thermal radiator. As the mechanical oscillations decay, pressure and/or density fluctuations are produced, for example, in the form of a soundwave. Such is detected as a measurement signal by means of a receiving unit, for example, an acoustic receiving unit, for example, a microphone, or using a flow sensor. An overview of photoacoustic gas analysis and measurements technology is presented, for example, in the scientific article, “Use of Acoustic Resonators in Photoacoustic Trace Gaseous Analysis and Metrology” by András Biklós, et al., Review of Scientific Instruments 72, 1937 (2001); doi: 10.1063/1.1353198.

For the spectroscopic measurement of gases with a broadband light source as emitter unit, a spectrally selective detection is necessary. Used for this are, besides optical filters also so-called photoacoustic, two chamber systems. In such case, a detection cell is filled with a reference gas, wherein the reference gas is the gas to be detected. Such a detector was invented by E. Lehrer and K. F. Luft in 1938 and a patent granted, DRP 730478, with the title “Verfahren zur Bestimmung von Bestandteilen in Stoffgemischen mittels Strahlungsabsorption” (“Method for Determining Components in Substance Mixtures by Means of Radiation Absorption”). The so-called “Luft detector” required large optical power, since the photoacoustic effect depends linearly on the radiated power. In the case of application of thermal radiators as emitter for infrared radiation, often mechanical choppers are applied for modulating the intensity. Such gas sensors have the disadvantage of not being very robust.

A MEMS based photoacoustic detection cell for a gas sensor is described in WO 20 210 380 99 A1. In such case, the detection cell is formed in a multi-ply substrate by means of a MEMS method of production.

Thus, there is a need for a low-energy, convenient and robust solution for gas detection.

### SUMMARY

The object is achieved by a gas sensor for determining concentration of at least one gas in a gas mixture as well as by a method for determining concentration of at least one gas in a gas mixture using a gas sensor according to the present disclosure.

Regarding the gas sensor, the object is achieved by a gas sensor for determining concentration of at least one gas in a gas mixture, comprising:
at least one intensity modulatable light source, which is especially selected from one of the following: a light emitting diode (LED), especially an infrared light emitting diode (IR-LED) or a UV light emitting diode (UV-LED), a laser diode, an organic light emitting diode (OLED), and a light emitting diode with optical resonator (RCLED);
a measuring section, into which the gas mixture to be investigated can be allowed to flow;
an essentially gas-sealed detection cell having an optical window, wherein the detection cell forms an acoustic resonator and a reference gas mixture containing at least one reference gas is filled into the detection cell;
a detection unit, which is embodied to register pressure and/or density fluctuations in the detection cell, and
an electronic data processing/evaluation unit, which is embodied based on the registered pressure and/or density fluctuations in the detection cell to determine the concentration of the at least one gas in the measuring section, wherein the gas sensor is embodied in such a manner that:
light emitted from the light source is radiated into the measuring section, wherein the intensity of the emitted light is modulated with a modulation frequency, which differs from the resonant frequency of a mode of the acoustic resonance of the detection cell by less than 0.5 times, especially less than 0.25 times, the half-width of the mode; and
the radiated light passes through the measuring section and then is radiated through the optical window into the first chamber.

The light source, the measuring section and the detection cell are arranged relative to one another, especially, along an optical path, for example, together with the light cable and/or optical elements serving for focusing.

The spectrum of the light source is especially selected in such a manner that it corresponds to the preferred field of application of the photoresonant, or photoacoustic, gas detection. The light source, thus its spectrum, is matched, in such case, to the gas to be detected. The light source is, thus, especially adapted to emit light of a frequency range, which is selected in such a manner that upon absorption of the transmitted light by the gas to be detected the gas to be detected is excitable to execute mechanical (e.g., molecular) oscillations.

For example, in an embodiment for which the gas to be detected is a gas excitable by infrared radiation of the light source, such that mechanical oscillations are executed, the light source is correspondingly an infrared light source. An infrared light source refers to an apparatus, which emits electromagnetic radiation in a wavelength range in the infrared (IR) region, especially with wavelengths greater than 700 nanometers (nm). Examples of gases, whose mechanical, molecular oscillations are excitable by infrared radiation, are CH₄, H₂O and CO₂.

In another embodiment, the gas to be detected is a gas excitable to mechanical oscillations by ultraviolet radiation of the light source, and the light source is correspondingly an ultraviolet light source. For example, mechanical, molecular oscillations in NO₂ are excitable by means of electromagnetic radiation in the ultraviolet region (UV).

The mechanical (molecular) oscillations of the gas are especially rotation and/or vibration oscillations. Especially, the gas to be detected is excited exclusively to mechanical (molecular) oscillations and, for example, not to electronic transitions.

By absorption of part of the radiated light in the measuring section, in the case of presence of the at least one gas to be detected in the gas mixture, the gas is excited to execute mechanical oscillations. The higher the concentration of the at least one gas in the measuring section, the more absorption takes place there. A non-absorbed portion of the radiated light is then radiated through the window into the detection cell. Since the reference gas mixture containing the at least one reference gas is located in the detection cell, also the reference gas in the detection cell is excited to execute mechanical oscillations.

Especially, the reference gas in the gas mixture is the at least one gas to be detected, whose concentration is detectable with the gas sensor.

In given cases, added to the gas mixture are other gases not excitable to execute mechanical oscillations by the light source. These serve as buffer gases. Examples of these are noble gases, for example, argon, and nitrogen.

The mechanical oscillations of the at least one reference gas in the detection cell bring about, similarly to the situation in the measuring section, pressure and/or density fluctuations as the oscillations decay. Because the detection cell is embodied as an acoustic resonator, the pressure and/or density fluctuations are correspondingly amplified by resonance effects.

In the context of the present disclosure, the detection cell is formed as an acoustic resonator; this being achieved especially by means of a corresponding dimensioning of the detection cell.

The more absorption takes place in the measuring section, the less there will be in the detection cell. Therefore, with increasing concentration of the at least one gas in the gas mixture, the pressure and/or density fluctuations registered by the detection unit decrease. The pressure and/or density fluctuations in the detection cell serve, thus, as the measurement signal to be processed by the data processing/evaluation unit. In at least one embodiment, the detection unit for detecting the pressure and/or density fluctuations is especially arranged in the interior of the detection cell.

Advantages of the gas sensor according to the present disclosure include the following:
An intensity modulatable light source is used. Especially, the intensity is sufficiently quickly modulatable, for example, the intensity is modulatable with a modulation frequency of at least 1000 Hz.
Corresponding light sources are provided, for example, by means of modern LEDs, especially infrared or ultraviolet LEDs. These have the advantage that they are cost effective and broadband. However, such have very low optical powers, for example, in the μW/mW range. As a result, they are actually not powerful enough for use in photoresonant and/or photoacoustic applications.
According to the present disclosure, the gas sensor is, however, embodied in such a manner that the intensity of the emitted light is modulated with a modulation frequency, which is adapted to the acoustic resonant frequency of the detection cell. This is because the modulation frequency deviates from the resonance frequency of a mode of the acoustic resonance of the detection cell by less than 0.5 times, in particular less than 0.25 times, the half-width of the mode. Preferably, the modulation frequency differs from the resonant frequency by less than 0.15 times the half-width of the mode. For typical applications, the modulation frequency differs from the resonant frequency, for example, by less than 500 Hz.
As a result of this adapting, the pressure and/or density fluctuations are sufficiently amplified to serve as a measurement signal for the gas concentration. The acoustically resonant detection is, thus, combined with an, especially, sufficiently fast, modulatable light source, in the case of which light source the optical power is, in given cases, significantly reducible.

In an embodiment of the gas sensor, the detection cell includes:
at least a first chamber having the optical window; and
a guide channel leading away from the first chamber,
wherein a cross-sectional area of the detection cell in the guide channel is reduced compared with the cross-sectional area of the first chamber.

By appropriate dimensioning of first chamber and the reduced guide channel, the detection cell is embodied as the acoustic resonator, especially according to the principle of a Helmholtz resonator.

In an embodiment of the gas sensor, the detection cell has a second chamber and the guide channel extends between the first chamber and the second chamber, wherein the first chamber communicates with the second chamber especially exclusively by means of the guide channel, and wherein especially the volume of the second chamber essentially equals the volume of the first chamber.

In an embodiment of the gas sensor, the detection unit has an acoustic receiving unit for detecting soundwaves produced in the detection cell.

The acoustic receiving unit is formed, for example, as a MEMS microphone. Preferably, the acoustic receiving unit is arranged in the second chamber, in case present.

In an embodiment of the gas sensor, the detection unit has a flow sensor, which is embodied to determine flow of the reference gas mixture in the guide channel. Serving as measure for the flow is, for example, mass flow rate, volume flow rate and/or flow velocity.

Preferably, the flow sensor is arranged in the guide channel, or between the first and the second chamber, in case present.

Preferably, the flow sensor is a MEMS flow sensor.

In an embodiment of the gas sensor, the gas sensor has at least two intensity modulatable light sources serving for exciting oscillations in mutually differing gases, which light sources are especially selected from one of the following: a light emitting diode (LED), especially an infrared light emitting diode (IR-LED) or a UV light emitting diode (UV-LED), a laser diode, an organic light emitting diode (OLED), a light emitting diode with optical resonator (RCLED).

By using at least two different light sources, different gases are excitable to execute mechanical oscillations, for example, CH₄ and CO₂. The plurality of light sources is, for example, especially formed compactly as an LED array.

In an embodiment of the gas sensor, the reference gas mixture contains at least two different reference gases.

In embodiments including a plurality of reference gases and light sources, the gas sensor serves as a multi-gas sensor, i.e., a gas sensor with which at least the concentration of two different gases is determinable.

In an embodiment of the gas sensor, the electronic data processing/evaluation unit has at least one frequency selective amplifier.

The frequency selective amplifier serves for better separation of the measurement signals for the case of a multi-gas sensor, so that the measurement signal for the concentration is safely assignable to a given gas. The frequency selective amplifier is, for example, an FPGA and/or an analog lock-in amplifier.

Regarding the method, the object is achieved by a method for determining concentration of at least one gas in a gas mixture with a gas sensor of the present disclosure, comprising steps as follows:
emitting light, wherein the intensity of the emitted light is modulated with a modulation frequency, which differs from the resonant frequency of a mode of the acoustic resonance of the detection cell by less than 0.5 times, especially less than 0.25 times, the half-width of the mode;
radiating the emitted light into the measuring section, which is passed through by the radiated light, wherein in the case of presence of the at least one gas in the gas mixture, the at least one gas is excited in the measuring section to execute mechanical oscillations by absorption of a part of the radiated light;
radiating a portion of the light non-absorbed in the measuring section through the optical window into the first chamber, wherein by absorption in the detection cell the at least one reference gas is excited to execute mechanical oscillations in the detection cell;
registering pressure and/or density fluctuations caused in the detection cell as a measurement signal, wherein the pressure and/or density fluctuations are caused by the mechanical oscillations of the at least one reference gas in the detection cell;
transmitting the pressure and/or density fluctuations registered by the detection unit to the data processing/evaluation unit;
determining the concentration of the at least one gas by the data processing/evaluation unit, based on the pressure and/or density fluctuations present in the detection cell.

Preferably, the modulation frequency differs from the resonant frequency by less than 0.15 or times the half-width of the mode.

In an embodiment, the method comprises steps as follows:
modulating the intensity of the radiated light of the first light source with a first modulation frequency;
modulating the intensity of the radiated light of the second light source with a second modulation frequency, wherein the first modulation frequency differs from the second modulation frequency;
assigning a determined concentration to a gas based on the pressure and/or density fluctuation caused by the gas by means of a frequency-based analysis.

In the aforementioned embodiment of the method, thus, the concentration of a plurality of gases is determinable, such that the gas sensor is a multi-gas sensor. In such case, as already mentioned, the reference gas mixture contains a plurality of reference gases, namely at least those gases, whose concentration is determinable with the method of the present disclosure.

In an embodiment of the method, the first modulation frequency and the second modulation frequency differ from one another by at least 10 Hz.

In an embodiment of the method, the first modulation frequency and the second modulation frequency differ from the resonant frequency of the same acoustic mode, especially the acoustic fundamental mode, of the acoustic resonance of the detection cell by less than 0.5 times, especially less than 0.25 times, preferably less than 0.15 times, the half-width of the acoustic mode.

In an embodiment of the method, the first modulation frequency differs from the resonant frequency of a first acoustic mode, especially the acoustic fundamental mode, of the acoustic resonance of the detection cell by less than 0.5 times, especially less than 0.25 times, preferably less than 0.15 times, the half-width of the first acoustic mode, wherein the second modulation frequency differs from the resonant frequency of a second acoustic mode of the acoustic resonance of the detection cell by less than 0.5 times, especially less than 0.25 times, preferably less than 0.15 times, the half-width of the second acoustic mode, and wherein especially the second modulation frequency is greater than the first modulation frequency.

In an embodiment, the method includes a step of applying a frequency selective amplification before the frequency-based analysis.

### BRIEF DESCRIPTION OF THE DRAWINGS

The gas sensor and method according to the present disclosure will now be explained in greater detail based on the appended, schematic drawings, wherein equal reference characters refer to equal features. When perspicuity requires or it otherwise appears sensible, reference characters already shown in earlier figures are omitted in subsequent figures.

The figures of the drawing show as follows:

FIG. 1a shows a first embodiment of the gas sensor according to the present disclosure;

FIG. 1b shows a second embodiment of the gas sensor in the form of a multi-gas sensor according to the present disclosure;

FIG. 2 shows an embodiment of the method according to the present disclosure;

FIG. 3 shows an example of a frequency selective analysis of an embodiment of the method with a multi-gas sensor according to the present disclosure.

### DETAILED DESCRIPTION

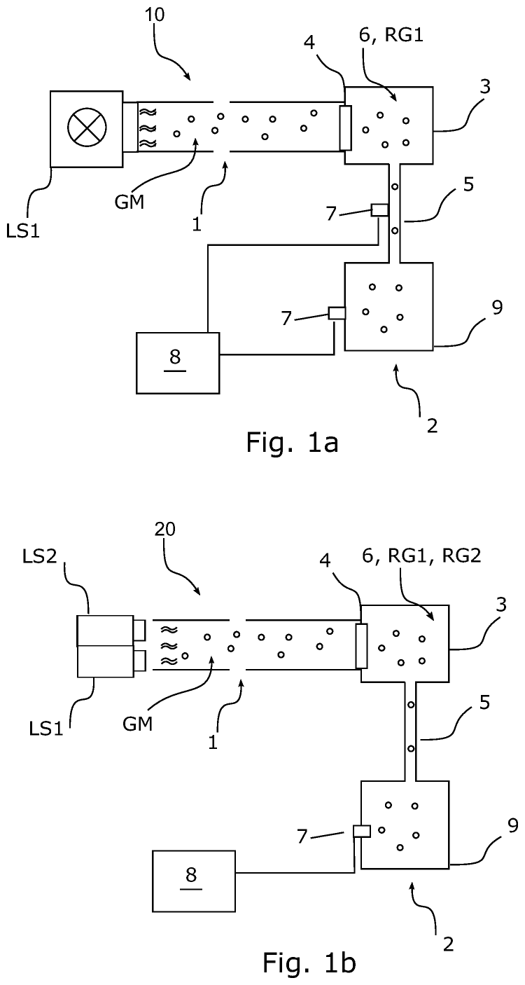

FIG. 1a shows an example of a gas sensor 10 of the present disclosure in a first embodiment. The gas sensor 10 includes a light source LS1, which radiates light into a measuring section 1 containing the gas mixture GM. Preferably, the light source is a cost effective, infrared light emitting diode (IR-LED) or UV light emitting diode (UV-LED), depending on the mechanical oscillation spectrum of the gas to be detected. For example, the light source may be an MIR-LED, e.g., an infrared light emitting diode, which emits light in the middle region of the infrared spectrum. The light emitting diode as light source is a preferred embodiment for the entire present disclosure, independently of additional components of a particular embodiment.

The gas mixture GM flows into the measuring section 1, for example, via openings in the measuring section 1. Depending on concentration of the gas to be detected, more or less absorption of the radiated light takes place in the measuring section 1. Then, the non-absorbed portion of the infrared light is radiated via a window 4 into a first chamber 3 of a detection cell 2. Filled into the detection cell 2 is a reference gas mixture 6 containing the at least one reference gas RG1 to be detected.

The detection cell 2 is sealed gas-tightly. The terminology, “gas-tightly”, means according to the present disclosure that the detection cell 2 is sufficiently sealed for the specific application of the photoacoustic detection with reference gases. This means, for example, that, for the one or more reference gases RG1, RG2 and the detection cell 2, a specific, average diffusion rate, which describes diffusion processes of reference gases RG1, RG2 into and out of the detection cell 2, is at most so large that the volume concentration of the respective reference gas RG1, RG2 in 24h changes at most by 50 ppm.

The detection cell 2 is formed as an acoustic resonator.

For this, the detection cell 2 comprises, for example, the first chamber 3, on which a guide channel 5 follows and communicates with a second chamber 9. The embodiment, shown in such case, of the detection cell 2 with two chambers 3, 9 and an intermediately lying guide channel 5 is not essential for the present disclosure. Other embodiments of the detection cell 2 as acoustic resonator, for example, having only a first chamber 3, are, of course, possible. Absorption also occurs in the detection cell 2, and the greater the concentration of the gas to be detected in the gas mixture GM, the less non-absorbed light is still available in the detection cell 2 for absorption processes in the reference gas mixture 6 with the reference gas RG1. Also decay of absorption processes in the reference gas mixture 6 with the reference gas RG1 cause pressure and/or density fluctuations.

By embodiment of the detection cell 2 as an acoustic resonator (here according to the Helmholtz principle), the pressure and/or density fluctuations are amplified and are registrable as a measurement signal by a detection unit 7. Detection unit 7 is, for example, a MEMS microphone, which is preferably arranged in the second chamber 9, as in the present embodiment, and/or a MEMS flow sensor arranged in the guide channel 5. The measurement signal registered by the detection unit 7 is weaker with rising gas concentration of the gas to be detected in the measuring section 1. A data processing/evaluation unit 8 is connected to the detection unit 7 in such a manner that the measurement signals registered by the detection unit 7 go to the data processing/evaluation unit 8 for evaluation. The data processing/evaluation unit 8 includes for this corresponding hardware and software components, especially electronic components such as calculation engines (e.g., processors) and/or memory elements.

Typically, the first chamber 3 has a volume of 100 to 1000 mm³. The guide channel 5 is preferably elongated, i.e., its length is at least 3 times as long as its diameter in a cross-sectional area. The cross-sectional area of the guide channel 5 amounts to, for example, 0.2 to 30 mm², preferably 0.8 to 3.2 mm².

In the case of a two-chamber system, preferably the second chamber 9 is the same size as the first chamber 3. The cross-sectional area of the detection cell 2 in the guide channel 5 is especially at most ½, i.e., half, the size, especially at most ¼ the size, of the cross-sectional area of the first chamber 3. This is the case at least in the region where the guide channel 5 communicates with the first chamber 3. The guide channel is especially cylindrical. Typically, the pressure and/or density fluctuations occur primarily in the guide channel 5 and extend into the first chamber serving as buffer volume and, in case present, into the second chamber 9, for example, at least 0.6 times a radius of the guide channel 5.

According to the present disclosure, the intensity of the light emitted from the light source LS1 is modulated with a frequency, which is adapted to a resonant frequency of the acoustic resonance of the detection cell 2.

FIG. 1b shows a preferred embodiment of a gas sensor of the present disclosure in the form of a multi-gas sensor. The construction shown in FIG. 1b corresponds essentially to the embodiment shown in FIG. 1a, except that the gas sensor 20 has a first light source LS1 and a second light source LS2. Such are separately modulatable in their intensity. Preferably, the light sources LS1, LS2 are formed as an array of a plurality of LEDs. The reference gas mixture 6 contains, furthermore, at least two different reference gases RG1, RG2, which correspond to the two gases to be detected. In the embodiment shown in FIG. 1b, thus, the concentrations of both of the gases are determinable by the gas sensor 20. This is especially also the case when they are both present at the same time in the gas mixture.

Thus, each of the two light sources LS1, LS2 is modulated in intensity with its own modulation frequency fmod1, fmod2. The two modulation frequencies fmod1, fmod2 differ from one another by at least 10 Hz. For better separation of the measurement signals with the different gases, the data processing/evaluation unit 8 includes a frequency selective amplifier, such as, for example, a lock-in amplifier.

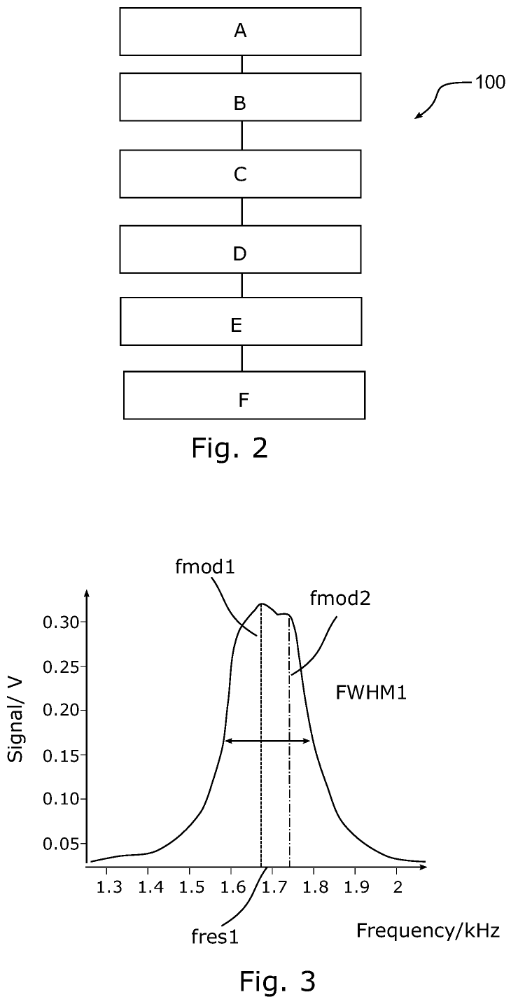

An embodiment of a method 100 of the present disclosure is shown in FIG. 2. The method 100 comprises in a step A the emitting of intensity modulated light modulated with a modulation frequency fmod1, fmod2 adapted to the acoustic resonance of the detection cell 2. The modulation frequency fmod1, fmod2 differs from the resonant frequency fres of a mode of the acoustic resonance of the detection cell 2 by less than 0.5 times, especially less than 0.25 times, the half-width FWHM1, FWHM2 of the mode.

Then, in a step B, the emitted light is radiated into the measuring section 1 and passes through the measuring section 1. In the case of presence in the gas mixture GM of the at least one gas to be detected, a portion of the radiated light is absorbed in the measuring section 1 and excites the gas in the measuring section 1 such that mechanical oscillations are executed.

Then, in a step C, a portion of the light not absorbed in the measuring section 1 is radiated through the optical window 4 into the first chamber 3 of the detection cell 2, in order there to excite the at least one reference gas RG1, RG2 such that mechanical oscillations are executed in the detection cell.

In a step D, the pressure and/or density fluctuations caused in the detection cell 2 are registered as a measurement signal. As explained above, the measurement signal decreases with rising concentration of the gas to be detected in the measuring section 1.

A step E includes the transmission of the pressure and/or density fluctuations registered by the detection unit 7 to the data processing/evaluation unit 8.

A step F includes determining the concentration of the at least one gas by the data processing/evaluation unit 8 based on the measurement signal, i.e., based on pressure and/or density fluctuations present in the detection cell 2.

FIG. 3 shows an example of evaluating the measurement signal using a frequency-based analysis in an embodiment of the method as multi-gas sensor. Plotted on the y-axis is the measurement signal registered by the detection unit 7, in such case, in the form of a registered electrical voltage. This is a function of frequency, which is plotted on the x-axis. The first acoustic resonance of the detection cell 2, e.g., the fundamental mode, lies at a first resonant frequency fres1.

In the example illustrated in such case, two modulation frequencies fmod1 and fmod2 are used, the first modulation frequency fmod1 (dashed line) for determining concentration of a first gas to be detected in the gas mixture, for example, CH₄, and the second modulation frequency fmod2 (dash-dotted line) for determining concentration of a second gas to be detected in the gas mixture, for example, CO₂. The modulation frequencies fmod1, fmod2 lie close to one another neighboring the same resonant frequency fres1 of the fundamental mode. Especially, the two modulation frequencies fmod1, fmod2 differ from the resonant frequency fres1 by less than 0.25 times the half-width FWHM1 of the fundamental mode.

Based on a frequency selective amplification, for example, by means of a correspondingly selected filtering degree and/or an integration time of an FPGA or a lock-lock-in amplifier, the peaks belonging to the different modulation frequencies fmod1, fmod2 can be better separated from one another in the data processing/evaluation unit 8. Preferably, a filtering degree of less than n=3, especially less than n=2, and an integration time of less than 10 seconds are used.

The two modulation frequencies fmod1, fmod2 lie preferably at least 10 Hz apart. In this way, concentrations determinable with the gas sensor 20 can be assigned to first and second gases. Alternatively to application of the same acoustic mode of the detection cell 2, it is also possible that the first modulation frequency fmod1, such as already shown in FIG. 3, lies at the peak of the resonant frequency fres1 of the fundamental mode, and the second modulation frequency fmod2 lies at the peak of a higher acoustic mode, i.e., a mode having a resonant frequency fres2 greater than the resonant frequency fres1 of the fundamental mode.

Embodiments of the present disclosure as multi-gas sensor have been explained using the example of two gases. Of course, the present disclosure is, however, not limited to this case. If the reference gas mixture 6 contains more than two reference gases RG1, RG2 to be detected and more than two individually intensity modulatable light sources LS1, LS2 are provided, then the concentrations of more than two mutually differing gases can be determined with the gas sensor of the present disclosure.

Claimed is:

1. A gas sensor for determining a concentration of at least one gas in a gas mixture, the gas sensor comprising:
at least one light source, which is operable to be intensity modulatable;
a measuring section, into which the gas mixture to be investigated is enabled to flow;
a substantially gas-sealed detection cell including an optical window, wherein the detection cell forms an acoustic resonator, and a reference gas mixture containing at least one reference gas is filled into the detection cell;
a detection unit, which is configured to register pressure and/or density fluctuations in the detection cell; and
an electronic data processing/evaluation unit, which is configured to determine the concentration of the at least one gas in the measuring section, based on the registered pressure and/or density fluctuations in the detection cell,
wherein the gas sensor is configured such that light emitted from the at least one light source is radiated into the measuring section, wherein at least a portion of the radiated light passes through the measuring section and then through the optical window into the detection cell, and
wherein an intensity of the emitted light is modulated with a modulation frequency which differs from a resonant frequency of a mode of an acoustic resonance of the detection cell by less than 0.5 times the half-width of the mode.

2. The gas sensor according to claim 1, wherein the detection cell includes:
at least a first chamber including the optical window, and
a guide channel extending away from the first chamber,
wherein a cross-sectional area of the guide channel is less than a cross-sectional area of the first chamber.

3. The gas sensor according to claim 2, wherein the detection cell includes a second chamber, and the guide channel extends between the first chamber and the second chamber,
wherein the first chamber communicates with the second chamber especially exclusively via the guide channel, and
wherein especially the volume of the second chamber essentially equals the volume of the first chamber.

4. The gas sensor according to claim 1, wherein the detection unit includes an acoustic receiving unit configured to detect soundwaves generated in the detection cell.

5. The gas sensor according to claim 1, wherein the detection unit includes a flow sensor configured to determine a flow of the reference gas mixture in the guide channel.

6. The gas sensor according to claim 1, wherein the at least one light source includes at least two light sources, each intensity modulatable and operable to excite mechanical oscillations in mutually differing gases in the gas mixture.

7. The gas sensor according to claim 1, wherein the at least one reference gas of the reference gas mixture includes at least two different reference gases.

8. The gas sensor according to claim 1, wherein the electronic data processing/evaluation unit includes at least one frequency selective amplifier.

9. The gas sensor according to claim 1, wherein the at least one light source is at least one of: a light emitting diode; an infrared light emitting diode or a UV light emitting diode; a laser diode; an organic light emitting diode; and a light emitting diode with optical resonator.

10. The gas sensor according to claim 1, wherein the intensity of the emitted light is modulated with a modulation frequency which differs from the resonant frequency of the mode of the acoustic resonance of the detection cell by less than 0.25 times the half-width of the mode.

11. A method for determining a concentration of at least one gas in a gas mixture using the gas sensor according to claim 1, the method comprising:
emitting light, wherein an intensity of the emitted light is modulated with a modulation frequency which differs from a resonant frequency of a mode of an acoustic resonance of the detection cell by less than 0.5 times, especially less than 0.25 times, the half-width of the mode;
radiating the emitted light into the measuring section, which is passed through by the radiated light, wherein, in the presence of the at least one gas in the gas mixture, the at least one gas in the measuring section is excited to execute mechanical oscillations by absorption of a portion of the radiated light;
radiating a portion of the light not absorbed in the measuring section through the optical window into the detection cell, wherein by absorption in the detection cell the at least one reference gas is excited to execute mechanical oscillations in the detection cell;
registering pressure and/or density fluctuations caused in the detection cell as a measurement signal, wherein the pressure and/or density fluctuations are caused by the mechanical oscillations of the at least one reference gas in the detection cell;
transmitting the measurement signal registered by the detection unit to the data processing/evaluation unit; and
determining the concentration of the at least one gas by the data processing/evaluation unit, based on the pressure and/or density fluctuations present in the detection cell.

12. The method according to claim 11, wherein the at least one light source includes a first light source and a second light source, each intensity modulatable and operable to excite mechanical oscillations in mutually differing gases in the gas mixture, the method further comprising:
modulating the intensity of the emitted light of the first light source with a first modulation frequency;
modulating the intensity of the emitted light of the second light source with a second modulation frequency, wherein the first modulation frequency differs from the second modulation frequency;
assigning a determined concentration to a gas based on the pressure and/or density fluctuation caused by the gas using a frequency-based analysis.

13. The method according to claim 12, wherein the first modulation frequency and the second modulation frequency differ from one another by at least 10 Hz.

14. The method according to claim 12, wherein the first modulation frequency and the second modulation frequency differ from the resonant frequency of the same acoustic mode, especially the acoustic fundamental mode, of the acoustic resonance of the detection cell by less than 0.5 times, especially less than 0.25, the half-width of the acoustic mode.

15. The method according to claim 12, wherein the first modulation frequency differs from the resonant frequency of a first acoustic mode, especially the acoustic fundamental mode, of the acoustic resonance of the detection cell by less than 0.5 times, especially less than 0.25 times, the half-width of the first acoustic mode,
wherein the second modulation frequency differs from the resonant frequency of a second acoustic mode of the acoustic resonance of the detection cell by less than 0.5 times, especially less than 0.25 times, the half-width of the second acoustic mode, and
wherein especially the second modulation frequency is greater than the first modulation frequency.

16. The method according to claim 12, further comprising:
applying a frequency selective amplification before the frequency-based analysis.
